(19) United States
(12) Patent Application Publication
(10) Pub. No.: US 2010/0057188 A1
(43) Pub. Date: Mar. 4, 2010

(54) ENDOPROSTHESES WITH POROUS REGIONS AND NON-POLYMERIC COATING

(75) Inventor: Jan Weber, Maastricht (NL)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/200,530

(22) Filed: Aug. 28, 2008

(51) Int. Cl. A61F 2/06

(52) U.S. Cl. 623/1.15; 623/1.44; 623/1.42; 623/1.46; 623/1.39

(57) ABSTRACT

Endoprostheses include an endoprosthesis wall that includes a surface layer that includes a metallic material and that defines a plurality of pores. A therapeutic agent fills one or more pores of the surface layer and a non-polymeric coating, covers the therapeutic agent in the one or more pores. The endoprostheses can, for example, deliver a therapeutic agent, such as a drug, in a controlled manner over an extended period of time.

### TECHNICAL FIELD

This invention relates to endoprostheses with porous regions and non-polymeric coating.

### BACKGROUND

The body includes various passageways such as arteries, other blood vessels and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In one aspect, the invention features an endoprosthesis that includes an endoprosthesis wall. The endoprosthesis wall includes a surface layer containing a metallic material and defining an irregular porous region, the pores in the porous region predominantly having a pore size of 25 nm or more, a therapeutic agent in one or more pores of the surface layer, and a non-polymeric bioresorbable coating covering the therapeutic agent in the one or more pores.

In another aspect, the invention features an endoprosthesis that includes an endoprosthesis wall. The endoprosthesis wall includes a surface layer comprising a metallic material and defining an irregular porous region, a therapeutic agent in one or more pores of the surface layer, and a non-polymeric bioresorbable coating covering the therapeutic agent in the one or more pores, in which the non-polymeric coating comprises a material selected from the group consisting of MgF₂, calcium phosphate, apatite, calcium carbonate, calcium fluoride, and mixtures thereof.

In another aspect, the invention features a method of making an endoprosthesis. The method includes providing an endoprosthesis preform comprising a metallic material, forming on a surface of the preform an irregular porous region, loading a therapeutic agent into one or more pores of the porous region, and depositing a non-polymeric bioresorbable coating onto the preform to cover the therapeutic agent such that the coating has a variable thickness or density across the surface.

Embodiments and/or aspects may include any one or more of the following features. The bioresorbable coating can be porous and the pores of the bioresorbable coating can have a pore size of about 50 nm or less, e.g., about 10 nm to about 50 nm. The non-polymeric coating can include a plurality of thinner regions and a plurality of thicker regions. The coating can have a surface morphology defined by the morphology of the surface layer. The thickness of the coating can be equal to the pore size or less. The bioresorbable coating can have regions of varying density covering different areas of the surface layer. The non-polymeric coating can have a mass density of about 0.2 to about 10.0 g/cm³. The non-polymeric coating can have a mass density of about 0.25 to about 5.0 g/cm3. The non-polymeric coating can include a material selected from the group consisting of MgF₂, calcium phosphate, apatite, calcium carbonate, calcium fluoride, and mixtures thereof. The pores can an average depth of about 10 nm to about 500 nm. The endoprosthesis body can include stainless steel.

Embodiments and/or aspects may also include any one or more of the following features. The coating can be deposited by IBAD, PLD, or PVD. The porous region can be formed by ion bombardment or dealloying. A therapeutic agent can be loaded by applying the therapeutic agent to the porous region in a solvent. The therapeutic agent can be loaded free of any non-therapeutic polymer carrier. The pores in the porous region can predominantly have a size of about 50 nm or more, for example, about 1 micron.

Embodiments and/or aspects may include any one or more of the following advantages. A stent can be provided that delivers a drug without the use of a polymer coating on the stent surface. The stent surface can be treated to form a porous matrix which acts as a drug reservoir. For example, the metal surface of a stent can be treated by dealloying to create large voids and pores, e.g. about 50 nm or more in cross-section. The large pores provide large volume cavities in which a substantial amount of drug can be readily incorporated. The rate of drug release from the pores is controlled by a non-polymeric, bioresorbable film, e.g. a magnesium, iron or calcium salt, which is deposited by a low-temperature process, e.g. ion beam assisted deposition (IBAD) or pulsed laser deposition (PLD), over and into the pores. The film can be porous, with much smaller pores of, e.g. 10 nm or less, which meters the delivery of drugs from the reservoir. Alternatively or in addition, the dissolution of the film exposes the underlying drug for release. Due to the irregular surface of the porous substrate, deposition of the film can result in variable thickness across the surface. Dissolution of the variable thickness film exposes drug over extended periods of time as a function of thickness. In addition, the density, and hence the dissolution rate of the coating can be varied in different regions over the stent. In embodiments, the stent is free of any non-therapeutic polymer, such as a polymer carrier for a therapeutic agent.

All publications, patent applications, patents, and other references mentioned herein are incorporated by reference herein in their entirety.

Other features and advantages of the invention will be apparent from the following detailed description, and from the claims.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIGS. 3A, 3C and 3D are longitudinal cross-sectional views of a stent wall, while FIG. 3B is a cross-sectional view of a stent wall skeleton with therapeutic agent and overcoating removed.

FIG. 4A-4D are longitudinal cross-sectional views of a stent wall, illustrating a method for making a stent.

FIG. 5 is a photograph of a porous region on a stent surface.

### DETAILED DESCRIPTION

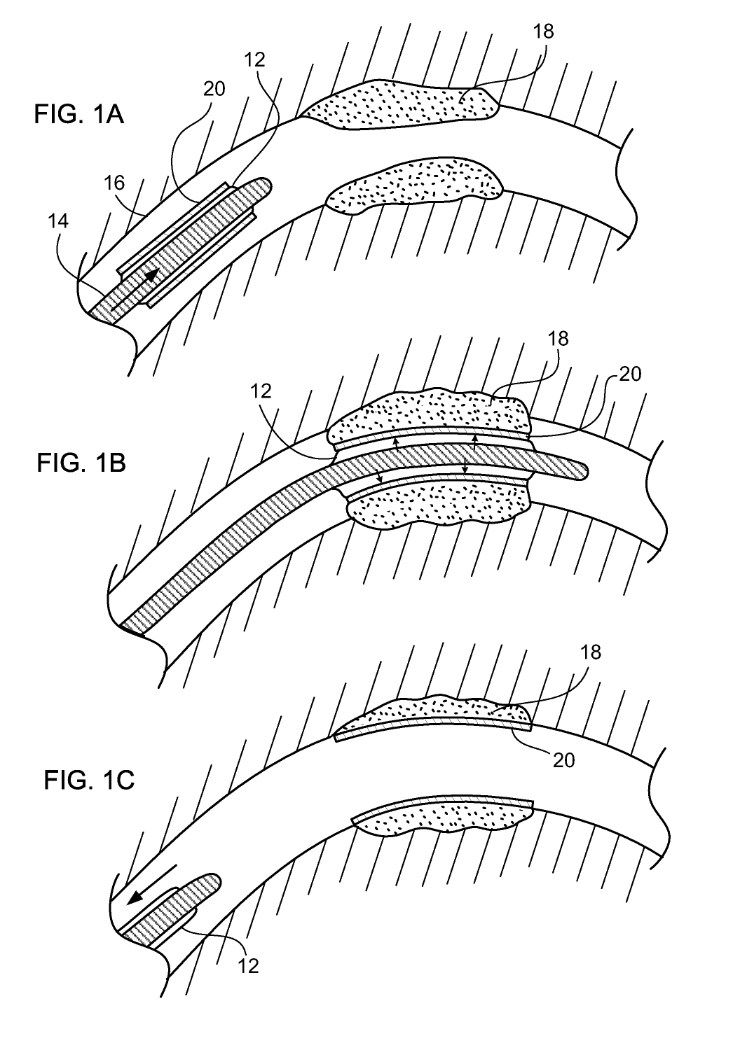

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

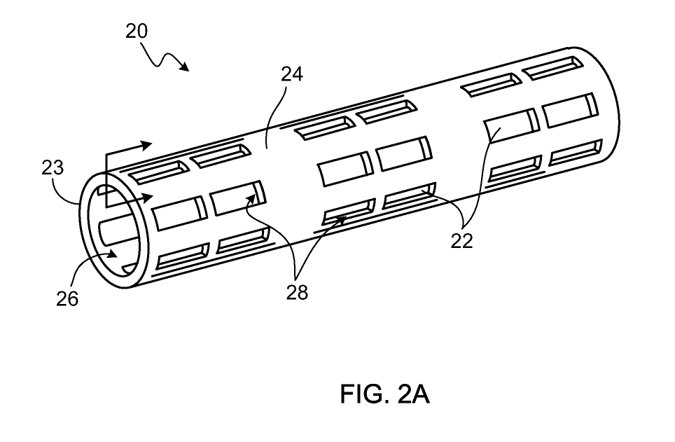

Referring to FIG. 2, the stent 20 includes a plurality of fenestrations 22 defined in a wall 23. Stent 20 includes several surface regions, including an outer, or abluminal, surface 24, an inner, luminal, surface 26, and a plurality of cutface surfaces 28. The stent can be balloon expandable, as illustrated above, or a self-expanding stent. Examples of stents are described in Heath '721, supra.

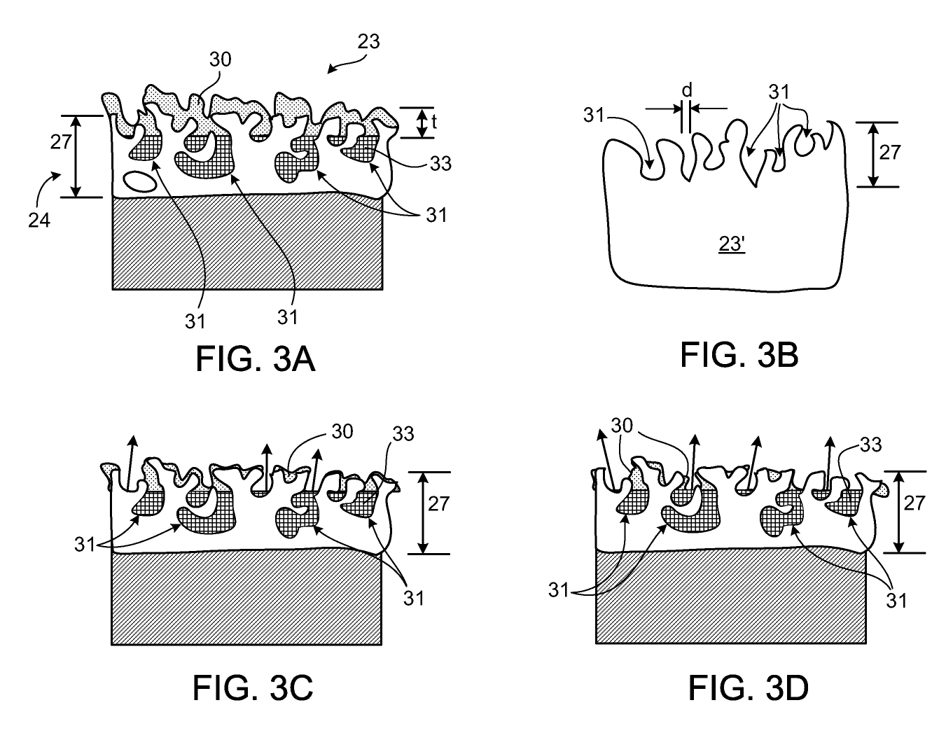

Referring to FIG. 3A, stent wall 23 has an abluminal surface layer 24 made of a metallic material. Surface layer 24 defines a plurality of irregular pores 31 (or voids) in a porous region 27. One or more therapeutic agents 33, such as a drug, is stored in one or more pores. A coating 30, such as one that includes a non-polymeric, bioresorbable material (e.g., MgF₂), covers the therapeutic agents 33. Such a construction can be utilized to deliver the one or more therapeutic agents to a body or a body lumen in a controlled manner and over an extended period of time, as will be further discussed below. Numerous small voids defined in the surface layer 24 can accommodate relatively large quantities of therapeutic agents, and since the voids are generally small in size, their presence generally does not typically degrade the mechanical properties of the stent, even when the stent has a relatively thin cross-section. Such a construction can, e.g., also reduce the likelihood of premature release of excessive amounts of the one or more therapeutic agents to the body or body lumen.

FIG. 3B, which is a cross-sectional view, shows the stent wall skeleton 23′ with the coating 30 and agents 33 removed, showing the pores or voids that can act as reservoirs for therapeutic agents. In embodiments, the pores extend into wall skeleton 23′ to a depth of about a few nanometers to about a few hundred nanometers, e.g., from about 10 nm to about 500 nm, from about 10 nm to about 350 nm or about 15 nm to about 200 nm. In embodiments, a maximum cross-sectional opening d of the pores, measured between adjacent walls of corresponding projections that define a pore, is between about 1 nm and about 1000 nm, e.g., between about 10 nm and about 800 nm or between about 50 nm and about 500 nm. In particular embodiments, a skeletal porosity, (the ratio of the void volume to total volume of solid and void with the therapeutic agent and coating removed from the stent) is greater than about 30%, e.g., greater than about 50%, greater than about 60% or greater than about 70%. In embodiments, substantially all of the therapeutic agents on the stent are housed within one of the defined pores. For example, in embodiments, more than 70% or more, e.g., 80% or more, or 90% or more, of the therapeutic agents are fully or partially within a void. This can provide drug reservoirs for a long-term slow release of the therapeutic agent from the stent when exposed to a biological environment. Each pore or void can be fully or partially filled with the therapeutic agents. In embodiments, the pores predominantly have a pore size that is relatively large, e.g. about 25 nm or 50 nm or more, which facilitates loading the drug into the pores and provides large pore volume in which a substantial quantity of drug can be stored.

Referring back now to FIG. 3A, in embodiments, coating 30 covers substantially all of the pores or voids filled with the therapeutic agent. In embodiments, the coating 30 has an average thickness (T) of from about 10 nm to about 800 nm, e.g., from about 10 to about 500 nm or from about 25 nm to about 250 nm. In embodiments, coating 30 does not have a constant thickness. Due to the porous feature of the region that coating 30 is deposited on, the thickness of the coating varies at different locations. In particular embodiments, the thickness of coating 30 on top of various drug filled pores varies. The thickness variations can be controlled by the shadowing and other effects of the irregular surface morphology during the deposition of the coating. In embodiments, the overall thickness of the coating is such that it follows the porous surface morphology and is not so great as to form a smooth uniform coating. In embodiments, the thickness of the coating is about the size of the pore openings or less.

Coating 30 is bioresorbable. For example, the coating can include MgF₂, calcium phosphate, apatite, calcium carbonate, calcium fluoride or mixtures of any of these materials. In embodiments, coating 30 has a mass density of about 1.0 g/cm³ to about 10.0 g/cm³, e.g., from about 1.5 g/cm³ to about 8.0 g/cm³ or from about 2.0 g/cm³ to about 4.0 g/cm³, measured prior to exposure to a biological fluid.

In embodiments, the coating is porous. For example, the pores can be nano-sized pores. In embodiments, the pores of the coating are from about 0.1 nm to about 50 nm, e.g., from about 0.2 nm to about 40 nm or from about 0.5 nm to about 30 nm. In embodiments, the volume percentage of the pores in the coating is from about 0.5% to about 90%, e.g., from about 1% to about 80% . In embodiments, at least some of the pores within the coating communicate with and/or are interconnected with pores of the porous region. In such embodiments, the interconnected pores can form channels, e.g., nano-sized channels to provide passageways for a therapeutic agent to be delivered to a body or body lumen. The porosity of the coating and the rate of erosion of the coating can be varied by varying the mass density of the coating.

To provide a long-term therapeutic benefit after the endoprosthesis is implanted in a human body, release of the one or more therapeutic agents at a therapeutic level over an extended period of time is desirable. Generally, a large volume of the therapeutic agent or blend of therapeutic agents utilized is stored in the stent, and the release rate is controlled by the properties of the porous region and/or coating, as described herein.

In embodiments, in which the coating is bioresorbable, the thickness of coating and the sizes of the pores defined in the coating change over time. In embodiments, the thickness of the coating decreases upon exposure to a biological fluid, expanding the pores of the porous region in a continuous manner. The eroded coating can facilitate an accelerated passage of therapeutic agents through the stent and to the body or body lumen. In some embodiments, with a portion of the coating fully eroded, the therapeutic agent is in contact with a biological fluid, a body or body lumen, such as a vascular wall. In embodiments, the thinner regions of coating 30 are fully eroded before the thicker regions of the coating, and the therapeutic agents the thinner regions of the coating covered obtain direct contact with a body fluid before the therapeutic agents that are covered by the thicker regions of coating 30. In embodiments, the thicker regions of coating 30 are fully eroded upon longer exposure of the coating to the biological fluid than the thinner regions. In embodiments, the therapeutic agents are exposed to the biological fluid on an extended time as a function of thickness.

In particular embodiments, the coating is or includes porous magnesium fluoride (MgF₂). For example, in embodiments, the MgF₂ coating erodes in the body or body lumen by, e.g., surface erosion processes. In embodiments and under human body conditions, an estimated erosion rate of the magnesium fluoride coating is from about 1 micron/year to about 50 microns/year e.g., from about 2 microns/year to about 30 microns/year or from about 5 microns/year to about 20 microns/year.

FIGS. 3A, 3C and 3D, which are cross-sectional views, show an exemplary stent as initially deployed (FIG. 3A) and over time after deployment of the stent to a lumen (FIGS. 3C and 3D). FIG. 3C shows a thinned coating (relative to that shown in FIG. 3A), while FIG. 3D shows an extensively eroded coating (relative to FIGS. 3A and 3C). FIG. 3C shows one or more regions of the coatings are fully eroded, exposing the underlying pores or voids that are filled with therapeutic agents directly to a body fluid. FIG. 3D shows regions that are more extensively eroded (relative to FIGS. 3A and 3C) that more therapeutic agents in the pores are directly exposed to a body fluid.

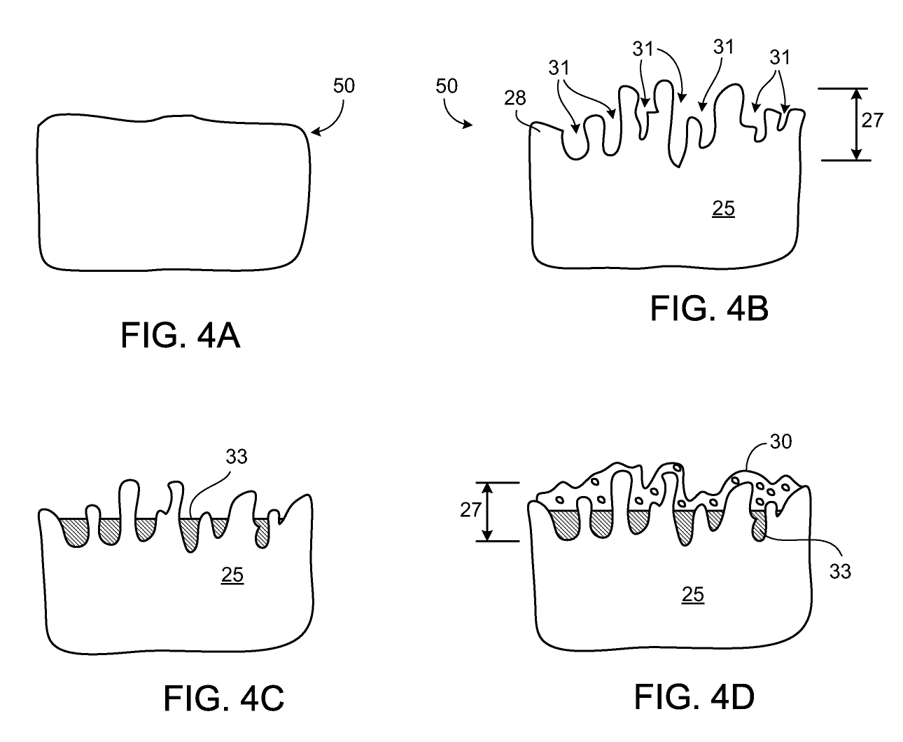

Referring to FIGS. 4A-4D, an exemplary process for making a stent is illustrated. To make a stent exemplified in FIG. 3A, a stent preform, such as a metal tube, is provided that includes a metallic material and that has a surface that defines a plurality of pores. For example, the porous stent preform can be provided by making porous a non-porous stent preform. Next, a therapeutic agent is deposited onto the preform in a manner that the therapeutic agent fills one or more pores of the porous preform. Finally, a coating that includes a non-polymeric material, such as magnesium fluoride, is deposited onto the therapeutic agent-filled preform to (at least partially) cover the therapeutic agent.

Referring particularly to FIGS. 4A and 4B, the non-porous stent preform 50, e.g., made of stainless steel, is made porous, such that surface 28 (e.g., an abluminal or luminal surface) defines a plurality of pores 31 (or voids). In embodiments, porous region 27 is made by etching the non-porous preform, such as by dealloying, glancing angle deposition, or laser ablation. Dealloying, which is sometimes called selective leaching, demetalification, or parting, is type of corrosion in alloys. In dealloying, a component of the alloy is preferentially leached from the material. Often the more electrochemically active component, usually a less noble metal, is the member that is selectively removed from the alloy by, what is believed to be, microscopic-scale galvanic corrosion, resulting in the formation of a porous sponge composed almost entirely of the more noble alloy constituents. Generally, the more susceptible alloys are the ones containing elements with a larger potential difference between each other in the galvanic series, e.g., copper and zinc in brass. An alloy may include any suitable combination of metals or combination of a metal and a non-metal, such as carbon and silicon. For example, the dealloying process may include dissolving one or more components of the alloy in a caustic substance. For example, a stainless steel alloy, or aluminum alloy can be dealloyed in sodium hydroxide solution, while a zinc alloy such as brass can be dealloyed in an acidic solution.

In certain embodiments, the dealloying process can be facilitated by applying electrical potential to the alloy in the caustic substance, e.g., by using an electrolytic cell. The structural morphologies of the porous region, such as porosity, pore size and pore depth can be selected by controlling dealloying conditions, such as concentration of the caustic substance, pH value, reaction temperature, electrical potential applied, and processing time. In general, higher reaction temperature and/or longer processing time produces higher porosity and larger pore size. The structural morphologies of the porous region can also be selected by controlling alloy composition in the surface region, as will be described further below. Dealloying techniques have been disclosed in Erlebacher et al., Nature 410, 450-453 (2001), Deakin et al., Corrosion Science, 46, 2117-2133 (2004), Senior et al., Nanotechnology, 17, 2311-2316 (2006), and Bayoumi et al., Electrochemistry Communications 8, 38-44 (2006).

Other selective etching techniques can be utilized. For example, preform 50 can first be modified by embedding some sacrificial components, such as particles of less noble metal, in a surface layer only and not alloying the entire body. Here, the sacrificial metal particles are then selectively removed or etched. The structural morphologies of the porous region can be selected by controlling the concentration of non-alloying sacrificial components embedded in the surface region. In certain embodiments, the porous region can include a ceramic, e.g., titania (“TiOx”), or alumina. In a particular embodiment, the porous region includes titania nanotubes formed by dealloying and anodic oxidation, as discussed in detail by Bayoumi et al., Electrochemistry Communication, 8, 38-44 (2006). Implantation can be effected, e.g., by plasma immersion ion implantation (“PIII”). For example, magnesium, aluminum, zinc, or other electrochemically more active metal can be implanted or embedded in a preform formed of stainless steel by metal plasma immersion ion implantation and deposition (“MPIIID”).

Referring particularly now to FIGS. 4B and 4C, after making the porous preform of FIG. 4B, one or more therapeutic agents is applied to the preform in a manner that the one or more therapeutic agents fills one or more pores of the preform. In particular embodiments, the one or more therapeutic agents are deposited without any non-therapeutic polymer or monomer carrier. The therapeutic agent can be deposited directly or by application in a suitable solvent. For example, the depositing can be effected by applying a potential difference, such as a few tens of millivolts to a few volts, between the porous region and the one or more therapeutic agents. In some embodiments, the one or more therapeutic agents are deposited into the porous region of the preform by dip coating or spraying the preform with a solution of the one or more agents in a solvent, followed by drying to remove any solvent under low temperature conditions, e.g., ambient conditions. The loading can be facilitated by repeatedly dipping and/or spraying. In other embodiments, the one or more therapeutic agents are deposited to the porous region by a vapor deposition process, such as PLD. The one or more therapeutic agents, can be deposited by providing drug as a target material in the PLD apparatus. In embodiments, about 25% or more, e.g., about 50 to 90% of the void volume of the porous region is occupied by one or more therapeutic agents or compositions that include the therapeutic agents.

Referring now particularly to FIGS. 4C and 4D, after the one or more therapeutic agents is deposited on the preform (preferably, in the pores of the preform), a non-polymeric coating that is erodible is deposited over the one or more therapeutic agents. In embodiments, the deposition of the non-polymeric coating, e.g., MgF₂, can be performed by, e.g., IBAD. The process IBAD includes a simultaneous thin film deposition or sputtering and directed ion bombardment of the film surface from an ion source.

For example, argon IBAD can be used for MgF₂ film deposition by electron beam evaporation of MgF₂ from a molybdenum crucible under a base pressure of, e.g., 1 E-05 Pa. The deposition rate of the film can vary in the rage of 0.1-2.0 nm/s or 0.3 to 1.5 nm/s, and the thickness of the film is about 1 to 800 nm, e.g., about 100 to about 200 nm. During this process, the stent, particularly the porous region, undergoes only a slight increase in temperature. In embodiments, the porous region temperature is less than 70° C., or at room temperature, when the argon ion bombardment energy is less than, e.g., 170 eV. The moderate temperature range of the deposition process is preferable because the one or more therapeutic agents are less likely to decompose at lower temperatures. In embodiments, the density of the coating can be varied by controlling the deposition conditions. Coatings of different density can be applied to different stent regions (e.g. by selective masking). More details of IBAD and argon IBAD deposition of MgF₂ can be found in Hirvonen et al., Materials and Processes for Surface and Interface Engineering, NATO-ASI Series, Series E: Applied Sciences, vol. 290, p. 307 (1995), and Dumas et al., Thin Solid Films 382, 61-68 (2001). In other embodiments, the coating can be deposited by a physical vapor deposition (PVD) process, e.g. PLD or by a direct evaporation process. Suitable processes are described in U.S. Ser. No. 11/752,736, filed May 23, 2007 and U.S. Ser. No. 11/752,772, also filed May 23, 2007.

Optionally, an additional therapeutic agent can be loaded on film 30, which can be further covered by an additional bioresorbable coating (not shown in figures). In embodiments, the additional bioresorbable coating has a similar structure (e.g., porous) and properties (e.g., eroding rate) as bioresorbable coating 30. In embodiments, the additional therapeutic agent is released before therapeutic agent 33 and facilitates maintaining a longer therapeutic release. In embodiments, the additional therapeutic agent is the same as therapeutic agent 33. In other embodiments, the additional therapeutic agent is different from therapeutic agent 33. In such embodiments, multiple types of drugs are loaded, where the drugs can be released in a desired sequence by controlling their loading sequence.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); immunosuppressant (e.g., everolimus, rapamycin, and zotarolimus); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. In embodiments, the drug can be incorporated within the porous regions in a polymer coating. Polymers for drug elution coatings are also disclosed in U.S. Published Patent Application No. 2005/019265A. A functional molecule, e.g., an organic, drug, polymer, protein, DNA, and similar material can be incorporated into groves, pits, void spaces, and other features of the stent.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stents described herein can be configured for vascular, e.g., coronary and peripheral vasculature or non-vascular lumens. For example, they can be configured for use in the esophagus or the prostate. Other lumens include biliary lumens, hepatic lumens, pancreatic lumens, urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., see U.S. Pat. No. 6,290,721).

While embodiments have been described in which an entire surface region of a pre-stent or the skeleton of a stent is made porous, in some embodiments, only a portion of a surface region of a pre-stent or skeleton stent is made porous. In still other embodiments, all surface regions are made porous.

While embodiments have been described in which an entire surface region of a pre-stent or the skeleton of a stent includes a therapeutic agent, in some embodiments, only a portion of a surface region of a pre-stent or skeleton stent includes a therapeutic agent. In still other embodiments, all surface regions include a therapeutic agent.

The processes can be performed on other medical devices, such as guide wires, and filters.

### EXAMPLE

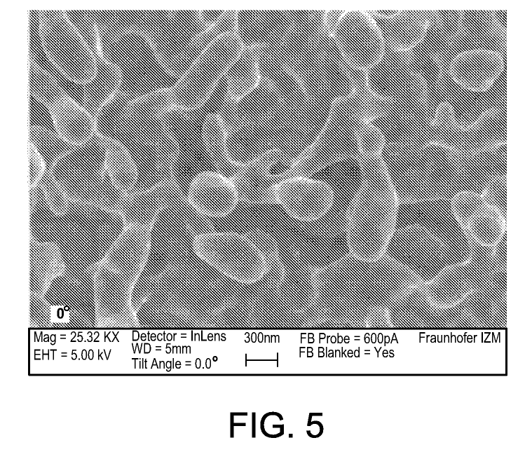

A porous region can be formed on a stent surface by argon plasma ion immersion implantation. The argon ion beam is set to have an pulse energy of about 35 KeV and a pulse frequency of about 600 Hz. The ions are implanted at a dose of about 20×10¹⁷ atoms/cm² onto a stent surface made of stainless steel 316L at a temperature of about 276° C. A pore region having a plurality of pores are created on the stent surface (FIG. 5). The pores can have a size up to about 1 micrometer.

Still other embodiments are in the following claims.

What is claimed is:

1. An endoprosthesis, comprising:
an endoprosthesis wall comprising
a surface layer comprising a metallic material and defining an irregular porous region, the pores in the porous region predominantly having a pore size of 25 nm or more;
a therapeutic agent in one or more pores of the surface layer; and
a non-polymeric bioresorbable coating covering the therapeutic agent in the one or more pores.

2. The endoprosthesis of claim 1, wherein the bioresorbable coating is porous and the pores of the bioresorbable coating have a pore size of about 50 nm or less.

3. The endoprosthesis of claim 1, wherein the non-polymeric coating comprises a plurality of thinner regions and a plurality of thicker regions.

4. The endoprosthesis of claim 1, wherein the coating has a surface morphology defined by the morphology of the surface layer.

5. The endoprosthesis of claim 1, wherein the thickness of the coating is equal to the pore size or less.

6. The endoprosthesis of claim 1, wherein the bioresorbable coating has regions of varying density covering different areas of the surface layer.

7. The endoprosthesis of claim 1, wherein the non-polymeric coating has a mass density of about 0.2 to about 10.0 g/cm³.

8. The endoprosthesis of claim 1, wherein the non-polymeric coating has a mass density of about 0.25 to about 5.0 g/cm3.

9. The endoprosthesis of claim 1, wherein the non-polymeric coating comprises a material selected from the group consisting of MgF₂, calcium phosphate, apatite, calcium carbonate, calcium fluoride, and mixtures thereof.

10. The endoprosthesis of claim 1, wherein the pores have an average depth of about 10 nm to about 500 nm.

11. The endoprosthesis of claim 1, wherein the endoprosthesis body comprises stainless steel.

12. An endoprosthesis, comprising:
an endoprosthesis wall comprising
a surface layer comprising a metallic material and defining an irregular porous region;
a therapeutic agent in one or more pores of the surface layer; and
a non-polymeric bioresorbable coating covering the therapeutic agent in the one or more pores, wherein the non-polymeric coating comprises a material selected from the group consisting of MgF₂, calcium phosphate, apatite, calcium carbonate, calcium fluoride, and mixtures thereof.

13. The endoprosthesis of claim 12, wherein the bioresorbable coating is porous and the pores of the bioresorbable coating have a pore size of about 10 nm to 50 nm.

14. The endoprosthesis of claim 13, wherein the thickness of the coating is equal to the pore size or less.

15. The endoprosthesis of claim 13, wherein the bioresorbable coating has regions of varying density covering different areas of the surface layer.

16. The endoprosthesis of claim 13, wherein the pores have an average depth of about 10 nm to about 500 nm.

17. The endoprosthesis of claim 13, wherein the endoprosthesis body comprises stainless steel.

18. A method of making an endoprosthesis, comprising:
providing an endoprosthesis preform comprising a metallic material;
forming on a surface of the preform an irregular porous region;
loading a therapeutic agent into one or more pores of the porous region; and
depositing a non-polymeric bioresorbable coating onto the preform to cover the therapeutic agent such that the coating has a variable thickness or density across the surface.

19. The method of claim 18, comprising depositing the coating by IBAD, PLD, or PVD.

20. The method of claim 18, comprising forming the porous region by ion bombardment or dealloying.
